(19) United States
(12) Patent Application Publication
(10) Pub. No.: US 2026/0335185 A1
(43) Pub. Date: Oct. 29, 2026

(54) NECK ACUPOINT RECOGNITION METHOD AND DEVICE, MASSAGE ROBOT, AND DIGITAL HUMAN

(71) Applicant: Jianghan University, Wuhan (CN)

(72) Inventors: Qiang He, Wuhan (CN); Meng Wang, Wuhan (CN); HongXing Zhang, Wuhan (CN); Qian Tu, Wuhan (CN)

(21) Appl. No.: 19/414,597

(22) Filed: Dec. 10, 2025

(30) Foreign Application Priority Data
Apr. 23, 2025 (CN) 202510510391X

(51) Int. Cl. A61H 39/02; A61H 39/04; G06V 40/10

(52) U.S. Cl. CPC A61H 39/02; A61H 39/04; A61H 2201/1659; G06V 40/10

(57) ABSTRACT

A neck acupoint recognition method and device, a massage robot, and a digital human are provided. The method includes: determining, for a neck image to be recognized, key points associated with horizontal and vertical cun and feature points associated with meridians, determining unit vertical cun and unit horizontal cun of the neck image, and determining a Ren meridian and a Du meridian of the neck image, where the key points include a Yintang acupoint; determining a laryngeal prominence of the neck image; determining a positional relationship between first-type neck acupoints and the laryngeal prominence, the Yintang acupoint, the Ren meridian and the Du meridian, and determining the first-type neck acupoints; determining a correlation between the first-type neck acupoints and second-type neck acupoints, and determining the second-type neck acupoints. The accuracy and efficiency of neck acupoint recognition are improved by eliminating the need for pre-calibration of the neck acupoints.

### TECHNICAL FIELD

The present disclosure relates to the technical field of acupoint recognition, and in particular, to a neck acupoint recognition method and device, a massage robot, and a digital human.

### BACKGROUND

With the widespread use of electronic products such as mobile phones and tablets, the cervical spine often remains static for prolonged periods, posing a significant threat to cervical health. Traditional Chinese therapeutic massage can address up to 95% of cervical spine issues. Massaging acupoints through pressing, kneading, tapping, compressing, and rubbing can effectively relieve neck muscle fatigue. This approach works to dredge the meridians and collaterals, promote qi and blood circulation, improve head and neck blood flow, and alleviate discomfort caused by cervical spondylosis. Acupoint massage is convenient and easy to perform, particularly suitable for daily health care massage. Currently, acupoint positioning is primarily performed manually by professional physicians and requires extensive professional training. This hinders both the ability of non-professionals to perform daily healthcare through acupoint massage and the automation of massage medical devices.

The prior patent CN111598949A discloses an automatic positioning method for acupoints in the human head and neck. This method requires training a model based on an image dataset with marked acupoints, through which the neck acupoints are directly obtained. In other words, the existing technique involves training a model that takes an image to be recognized as input and outputs the corresponding neck acupoints. The training process of the model relies on the accurate labeling of the neck acupoints. This poses the following technical issues: neck acupoint recognition can only be achieved through the second image set obtained by manual labeling. The labeling process is complex and time-consuming, and the accuracy of manual labeling directly affects the recognition performance of the trained model. Small errors often lead to low recognition accuracy.

Therefore, there is an urgent need to provide a neck acupoint recognition method and device, a massage robot, and a digital human to improve the accuracy and efficiency of neck acupoint recognition.

### SUMMARY

In view of the above, it is necessary to provide a neck acupoint recognition method and device, a massage robot, and a digital human to solve the technical problems of lower accuracy and efficiency of neck acupoint recognition caused by reliance on manual labeling in the prior art.

In a first aspect, the present disclosure provides a neck acupoint recognition method, comprising:
determining, for a neck image to be recognized, key points associated with horizontal and vertical cun and feature points associated with meridians, determining a unit vertical cun and a unit horizontal cun of the neck image based on the key points, and determining the Ren meridian and the Du meridian of the neck image based on the feature points, wherein the key points include the Yintang acupoint;
determining a laryngeal prominence of the neck image to be recognized;
determining a positional relationship between first-type neck acupoints and a set of anatomical landmarks, wherein the set of anatomical landmarks includes the laryngeal prominence, the Yintang acupoint, the Ren meridian, and the Du meridian, and determining locations of the first-type neck acupoints based on the laryngeal prominence, the Yintang acupoint, the Ren meridian, the Du meridian, the positional relationship, the unit vertical cun and the unit horizontal cun;
determining a geometric association between the first-type neck acupoints and second-type neck acupoints, and determining the second-type neck acupoints based on the geometric association and the first-type neck acupoints.

In some possible embodiments, the key points further include a left anterior hairline point and a right anterior hairline point that are associated with the horizontal cun, as well as an anterior hairline associated with the vertical cun; the feature points include a left auricular lobule point, a right auricular lobule point, a left acromion point and a right acromion point; determining the key points associated with horizontal and vertical cun and feature points associated with meridians of the neck image to be recognized, comprises:
detecting the neck image to be recognized to obtain a plurality of candidate key points; based on a mapping relationship between candidate key point numbers and the candidate key points,
determining the left anterior hairline point, the right anterior hairline point, a first target point and a second target point that are related to the Yintang acupoint, the left auricular lobule point, the right auricular lobule point, the left acromion point and the right acromion point from the plurality of candidate key points; determining the midpoint of the first target point and the second target point as the Yintang acupoint;
determining the rectangular facial box in the neck image to be recognized, and defining the upper boundary of the rectangular facial box as the anterior hairline.

In some possible embodiments, determining a unit vertical cun and a unit horizontal cun of the neck image based on the key points, and determining a Ren meridian and a Du meridian of the neck image based on the feature points, comprises:
determining a vertical distance and vertical bone cun between the Yintang acupoint and the anterior hairline, as well as a horizontal distance and horizontal bone cun between the left anterior hairline point and the right anterior hairline point;
determining the unit vertical cun based on the vertical distance and the vertical bone cun, and determining the unit horizontal cun based on the horizontal distance and the horizontal bone cun;
determining a first connecting line between the left and right auricular lobule points and a first midpoint of the first connecting line; and defining a first straight line that is perpendicular to the first connecting line and passes through the first midpoint on an anterior side of a human body as the Ren meridian; then, determining a second connecting line between the left and right acromion points and a second midpoint of the second connecting line; and defining a second straight line that is perpendicular to the second connecting line and passes through the second midpoint on a posterior side of the human body as the Du meridian.

In some possible embodiments, determining the laryngeal prominence of the neck image to be recognized, comprises:
determining a plurality of candidate reference points and a plurality of candidate reference point numbers of the neck image to be recognized;
determining the laryngeal prominence based on a laryngeal prominence number and a pre-built association relationship between candidate reference point numbers and candidate reference point positions.

In some possible embodiments, the positional relationship is bone-degree positions relationship; then, determining locations of the first-type neck acupoints based on the laryngeal prominence, the Yintang acupoint, the Ren meridian, the Du meridian, the positional relationship, the unit vertical cun and the unit horizontal cun, comprises:
determining bone-degree positions of the first-type neck acupoints based on the laryngeal prominence, the Yintang point, the Ren meridian, the Du meridian, and the positional relationship;
converting the bone-degree positions to image positions based on the unit vertical cun and the unit horizontal cun, and taking the image positions as positions where the first-type neck acupoints are located.

In some possible embodiments, the first-type neck acupoints include Renying, Futu, Tiantu and Tianchuang that are located on an anterior neck, as well as Yamen and Dazhui that are located on a posterior neck; where the positional relationship, comprises:
the Renying acupoint is 1.5 horizontal cun from the laryngeal prominence;
the Futu acupoint is 3 horizontal cun from the laryngeal prominence;
the Tiantu acupoint is located on the Ren meridian and below the laryngeal prominence and is 2 vertical cun from the laryngeal prominence;
the Tianchuang acupoint is 3.5 horizontal cun from the laryngeal prominence;
the Yamen acupoint is located on the Du meridian and is 14.5 vertical cun from the Yintang acupoint;
the Dazhui acupoint is located on the Du meridian and is 18 vertical cun from the Yintang acupoint.

In some possible embodiments, the second-type neck acupoints include Lianquan, Qishe, Shuitu, Quepen and Tianding on the anterior neck, as well as Fengfu, Fengchi, Jingbailao and Tianzhu on the posterior neck; where the geometric association is as follows:
the Lianquan acupoint is located at the midpoint of the line connecting the chin and the laryngeal prominence; where the chin is a lower box line of the rectangular facial box;
the Qishe acupoint is 1.5 horizontal cun from the Tiantu acupoint;
the Shuitu acupoint is located on the Ren meridian and at the midpoint of the line connecting the Qishe acupoint and the Renying acupoint;
the Quepen acupoint is the intersection point of the extension line of the line connecting the Tiantu acupoint and the Qishe acupoint and a target line segment, where the target line segment is the line segment that is perpendicular to the line connecting the left nipple and the right nipple and passes through the left nipple or the right nipple;
the Tianding acupoint is located at the midpoint of the line connecting the Futu acupoint and the Quepen acupoint;
the Fengfu acupoint is located above the Yamen acupoint and is 0.5 vertical cun from the Yamen acupoint;
the Fengchi acupoint is 1.3 horizontal cun from the Fengfu acupoint;
the Jingbailao acupoint is located above the Dazhui acupoint and is 2 vertical cun and 1 horizontal cun from the Dazhui acupoint;
the Tianzhu acupoint is located below the Yamen acupoint and is 0.3 vertical cun and 1.3 horizontal cun from the Yamen acupoint.

In another aspect, the present disclosure further provides a neck acupoint recognition device, comprising:
a reference information determination unit, configured to determine the key points associated with horizontal and vertical cun and feature points associated with meridians, of a neck image to be recognized, determine the unit vertical cun and the unit horizontal cun of the neck image based on the key points, and determine the Ren meridian and the Du meridian of the neck image based on the feature points, wherein the key points include the Yintang acupoint;
a laryngeal prominence determination unit, configured to determine a laryngeal prominence of the neck image to be recognized;
a first-type neck acupoint determination unit, configured to determine a positional relationship between the first-type neck acupoints and a set of anatomical landmarks, wherein the set of anatomical landmarks includes the laryngeal prominence, the Yintang acupoint, the Ren meridian, and the Du meridian, and determine locations of the first-type neck acupoints based on the laryngeal prominence, the Yintang acupoint, the Ren meridian, the Du meridian, the positional relationship, the unit vertical cun and the unit horizontal cun;
a second-type neck acupoint determination unit, configured to determine a geometric association between the first-type neck acupoints and second-type neck acupoints, and determine the second-type neck acupoints based on the geometric association and the first-type neck acupoints.

In another aspect, the present disclosure further provides a massage robot, including an acupoint recognition subsystem configured to determine neck acupoints and a massage subsystem configured to perform massage based on the neck acupoints, where the acupoint recognition subsystem includes a memory and a processor, where

the memory is configured to store a program;

the processor is coupled with the memory and configured to execute the program stored in the memory to implement the steps of the neck acupoint recognition method in any one of the above-mentioned possible embodiments.

In another aspect, the present disclosure further provides a digital human, including neck acupoints that are used for a virtual neck model and are marked in the virtual neck model, where the neck acupoints are determined based on a neck acupoint recognition method, where the neck acupoint recognition method is the neck acupoint recognition method in any one of the above-mentioned possible embodiments.

The beneficial effects of adopting the above-mentioned embodiments are as follows. By determining the positional relationship between the laryngeal prominence, the Yintang acupoint, the Ren meridian and the Du meridian and the first-type neck acupoints, the neck acupoint recognition method provided by the present disclosure achieves that the first-type neck acupoints can be recognized based on the positional relationship merely by determining the laryngeal prominence, the Yintang acupoint, the Ren meridian and the Du meridian. Then, the second-type neck acupoints can be determined based on the correlation relationship, thereby obtaining all neck acupoints. Compared to labeling of the neck acupoints, the laryngeal prominence is a more distinct landmark of the human body, and the key points and the Yintang acupoint, the Ren meridian and the Du meridian are also distinct, which makes the calibration process simpler. Also, there are various algorithms in the prior art that can directly determine the laryngeal prominence, the key points, and the feature points without the need for sample labeling and training processes. In other words, the localization process for the neck acupoints does not rely on manual labeling, ensuring the accuracy and efficiency of neck acupoint recognition. Also, the present disclosure constructs the unit vertical cun and the unit horizontal cun through the determined key points. Through the unit vertical cun and the unit horizontal cun, the impact of individual anatomical variations on neck acupoint localization is mitigated, further ensuring the accuracy of neck acupoint recognition.

### BRIEF DESCRIPTION OF THE DRAWINGS

The accompanying drawings described in the following embodiments are provided to clearly illustrate the technical solutions of the present disclosure. It will be understood that these drawings depict only certain embodiments of the present disclosure and are not to be considered limiting of its scope. For those skilled in the art, other drawings can also be obtained according to these drawings without creative efforts.

FIG. 1 is a schematic flowchart of an embodiment of a neck acupoint recognition method according to the present disclosure;

FIG. 2 is a schematic flowchart of an embodiment for determining the unit vertical cun and the unit horizontal cun according to the present disclosure;

FIG. 3 is a schematic flowchart of an embodiment of S102 in FIG. 1 according to the present disclosure;

FIG. 4 is a schematic diagram of an embodiment showing a detection result of the second detection algorithm according to the present disclosure;

FIG. 5 is a schematic flowchart of an embodiment of determining neck acupoints in S103 in FIG. 1 according to the present disclosure;

FIG. 6 is a schematic structural diagram of an embodiment of a neck acupoint recognition device according to the present disclosure;

FIG. 7 is a schematic structural diagram of an embodiment of a massage robot according to the present disclosure.

### DETAILED DESCRIPTION OF THE EMBODIMENTS

The technical solutions in the embodiments of the present disclosure will be clearly and completely described below in conjunction with the accompanying drawings in the embodiments of the present disclosure. It is evident that the described embodiments are merely some rather than all of the embodiments of the present disclosure. Based on the embodiments of the present disclosure, all other embodiments obtained by those skilled in the art without creative efforts fall within the scope of protection of the present disclosure.

It should be understood that the illustrative drawings are not drawn according to a scale of a real object. The flowcharts used in the present disclosure illustrate operations implemented according to some embodiments of the present disclosure. It should be understood that the operations of the flowcharts can be implemented out of order, and steps without a logical contextual relationship can be implemented in reverse order or implemented simultaneously. Additionally, those skilled in the art, under the guidance of the content of the present disclosure, can add one or more other operations to the flowcharts or can also remove one or more operations from the flowcharts. Some block diagrams shown in the drawings are functional entities and do not necessarily have to correspond to physically or logically separate entities. These functional entities may be implemented in software form, or implemented in one or more hardware modules or integrated circuits, or implemented in different networks and/or processor systems and/or microcontroller systems.

The descriptions of “first,” “second,” or the like, involved in the embodiments of the present disclosure are merely for descriptive purposes and cannot be understood as indicating or implying relative importance or implicitly specifying the quantity of indicated technical features. Therefore, the technical features defined by “first” and “second” may explicitly or implicitly include at least one such feature.

The reference to “embodiments” herein means that a particular feature, structure, or characteristic described in connection with the embodiments can be included in at least one embodiment of the present disclosure. The appearance of this phrase in various places in the specification does not necessarily refer to the same embodiment, or may not be an independent or candidate embodiment that is mutually exclusive of other embodiments. It is explicitly and implicitly understood by those skilled in the art that the embodiments described herein can be combined with other embodiments.

The present disclosure provides a neck acupoint recognition method and device, a massage robot, and a digital human, which are described separately below.

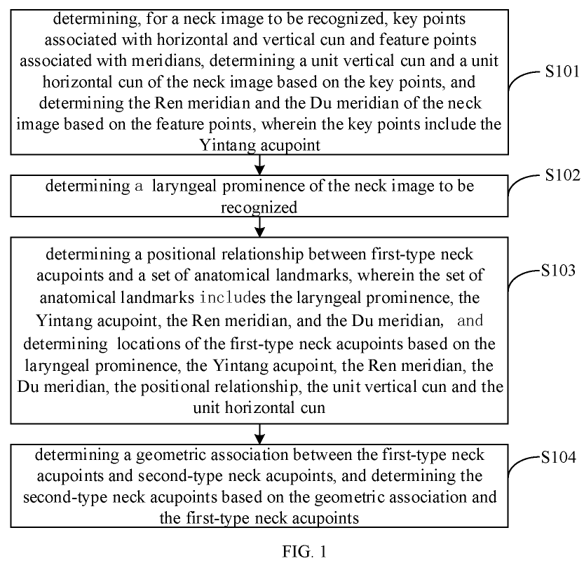

FIG. 1 is a schematic flowchart of an embodiment of a neck acupoint recognition method proposed in the present disclosure. As shown in FIG. 1, the neck acupoint recognition method comprises:

S101, determining, for a neck image to be recognized, key points associated with horizontal and vertical cun and feature points associated with meridians, determining a unit vertical cun and a unit horizontal cun of the neck image based on the key points, and determining the Ren meridian and the Du meridian of the neck image based on the feature points, wherein the key points include the Yintang acupoint.

The neck image to be recognized is acquired as follows: three-dimensional point cloud data of head and neck of a human body is collected based on a depth camera, and the three-dimensional point cloud data is taken as the neck image to be recognized.

In a specific embodiment of the present disclosure, the key points associated with the horizontal and vertical cun and the feature points associated with the meridians of the neck image to be recognized are determined using the first detection algorithm. The first detection algorithm is a mature algorithm in the existing technology. Specifically, an open-source algorithm that can be used directly without the need for training. Through this setting, the efficiency of neck acupoint recognition can be further improved.

The unit vertical cun refers to a conversion relationship between an image size and the bone-degree positions along a first direction, where the first direction is a direction from head to toe. The unit horizontal cun refers to a conversion relationship between an image size and a bone-degree positions along a second direction, where the second direction is perpendicular to the first direction.

It is to be noted that the "cun" in the embodiments of the present disclosure is not a commonly used measurement unit in daily life, but a unit of equal division on the body. For different individuals, the length of the unit of equal division is different.

It is also to be noted that the selection of the key points needs to be performed based on a bone-degree measurement method. For example, the bone-degree measurement method specifies the bone-degree distance between the anterior hairline and the posterior hairline, so the key points need to be the anterior hairline and the posterior hairline. In other words, the key points refer to points corresponding to the bone-degree measurements in the bone-degree measurement method.

S102, determining a laryngeal prominence of the neck image to be recognized.

Specifically, the laryngeal prominence of the neck image to be recognized is determined based on a second detection algorithm, where the second detection algorithm is also a mature algorithm in the prior art. Specifically, the second detection algorithm is an open-source algorithm that does not require training and can be used directly. Through this setup, the efficiency of neck acupoint recognition can be further improved.

S103, determining a positional relationship between first-type neck acupoints and a set of anatomical landmarks, wherein the set of anatomical landmarks includes the laryngeal prominence, the Yintang acupoint, the Ren meridian, and the Du meridian, and determining locations of the first-type neck acupoints based on the laryngeal prominence, the Yintang acupoint, the Ren meridian, the Du meridian, the positional relationship, the unit vertical cun and the unit horizontal cun.

The first-type neck acupoints refer to neck acupoints directly associated with the laryngeal prominence, the Yintang acupoint, the Ren meridian, and the Du meridian.

S104, determining a geometric association between the first-type neck acupoints and second-type neck acupoints, and determining the second-type neck acupoints based on the geometric association and the first-type neck acupoints.

The second-type neck acupoints refer to neck acupoints that are not directly associated with the laryngeal prominence, the Yintang acupoint, the Ren meridian and the Du meridian, but have a geometric association with the first-type neck acupoints.

It should be understood that the neck acupoint recognition method in the embodiments of the present disclosure can be implemented in any device that is based on neck acupoint recognition, for example, an acupuncture and moxibustion robot, a massage robot, or the like. Specifically, the neck acupoint recognition method is stored in the aforementioned device in the form of a programmed program. When the device is activated, the program is called, and the neck acupoint recognition method is implemented.

Compared with the prior art, by determining the positional relationship between the laryngeal prominence, the Yintang acupoint, the Ren meridian and the Du meridian and the first-type neck acupoints, the neck acupoint recognition method provided by the present disclosure achieves that the first-type neck acupoints can be recognized based on the positional relationship merely by determining the laryngeal prominence, the Yintang acupoint, the Ren meridian and the Du meridian. Then, the second-type neck acupoints can be determined based on the correlation relationship, thereby obtaining all neck acupoints. Compared to labeling of the neck acupoints, the laryngeal prominence is a distinctive landmark of the human body, and the key points and the Yintang acupoint, the Ren meridian and the Du meridian are also distinct, which makes the calibration process simpler. Also, there are various algorithms in the prior art that can directly determine the laryngeal prominence, the key points, and the feature points without the need for sample labeling and training processes. In other words, the localization process for the neck acupoints does not rely on manual labeling, which improves the accuracy and efficiency of neck acupoint recognition. Also, the embodiments of the present disclosure construct the unit vertical cun and the unit horizontal cun through the determined key points. Through the unit vertical cun and the unit horizontal cun, the impact of individual anatomical variations on neck acupoint localization is mitigated, further ensuring the accuracy of neck acupoint recognition.

In some embodiments of the present disclosure, the bone-degree measurements in the bone-degree measurement method are as follows: a horizontal bone-degree between the left anterior hairline point and the right anterior hairline point is 9 cun, and a vertical bone-degree between the anterior hairline and the Yintang acupoint is 3 cun. Correspondingly, the key points include the left anterior hairline point and the right anterior hairline point that are associated with the horizontal cun, as well as the anterior hairline associated with the vertical cun.

It should be understood that the bone-degree measurements are not limited to the above-mentioned specific embodiments. For example, the vertical bone-degree can also be the bone-degree between the anterior hairline and the posterior hairline, which will not be enumerated here.

The Ren meridian and the Du meridian are midlines of the front and back of the human body, respectively, and the human body is a symmetrical structure. Therefore, in some embodiments of the present disclosure, the feature points should be symmetrical points. Specifically, the feature points include a left auricular lobule point, a right auricular lobule point, a left acromion point and a right acromion point.

Based on the above-mentioned key points and feature points, in some embodiments of the present disclosure, the first detection algorithm includes a key point detection algorithm for determining the left anterior hairline point, the right anterior hairline point, the left auricular lobule point, the right auricular lobule point, the left acromion point and the right acromion point, and the Yintang acupoint, as well as a face detection algorithm for determining the anterior hairline. Then, determining the key points associated with horizontal and vertical cun and feature points associated with meridians of the neck image to be recognized, comprises in S101 comprises:
detecting the neck image to be recognized to obtain a plurality of candidate key points, and determining, based on a mapping relationship between candidate key point numbers and the candidate key points, the left anterior hairline point, the right anterior hairline point, a first target point and a second target point that are related to the Yintang acupoint, the left auricular lobule point, the right auricular lobule point, the left acromion point and the right acromion point from the plurality of candidate key points;
determining the midpoint of the first target point and the second target point as the Yintang acupoint;
determining the rectangular facial box in the neck image to be recognized, and defining the upper boundary of the rectangular facial box as the anterior hairline.

The neck image to be recognized can be detected based on the key point detection algorithm, and the rectangular facial box of the neck image to be recognized can be determined based on the face detection algorithm.

It is to be noted that a detection result of the key point detection algorithm includes the numbers of the candidate key points. In other words, when determining the left anterior hairline point, the right anterior hairline point, the left auricular lobule point, the right auricular lobule point, the left acromion point, the right acromion point, the first target point and the second target point, the left anterior hairline point, the right anterior hairline point, the left auricular lobule point, the right auricular lobule point, the left acromion point, the right acromion point, the first target point and the second target point can be quickly determined from the plurality of candidate key points merely according to the pre-determined numbers of the left anterior hairline point, the right anterior hairline point, the left auricular lobule point, the right auricular lobule point, the left acromion point, the right acromion point, the first target point and the second target point. This further improves the efficiency of neck acupoint recognition.

In a specific embodiment of the present disclosure, the key point detection algorithm is Mediapipe or Dlib.

The Mediapipe can extract 478 key points of a face, where key points 8 and 9 are the first target point and second target point, where the midpoint of the first target point and the second target point is the Yintang acupoint, where key points 103 and 332 are the left anterior hairline point and right anterior hairline point.

The Dlib can extract 68 key points of a race, including facial features such as eyes, eyebrows, nose, and mouth, where key points 21 and 22 are the first target point and second target point, where the midpoint of the first target point and the second target point is the Yintang acupoint, where key points 18 and 25 are the left anterior hairline point and right anterior hairline point.

The specific numbers of the left auricular lobule point, the right auricular lobule point, the left acromion point and the right acromion point are not elaborated here.

In a specific embodiment of the present disclosure, the face detection algorithm is a RetinaFace algorithm or a MogFace algorithm. Detection results of both the RetinaFace and MogFace algorithms include a detection box, where an upper box line of the detection box is aligned with the anterior hairline, and a lower box line is aligned with the chin.

The embodiments of the present disclosure can quickly determine the anterior hairline through the rectangular facial box obtained from the face detection algorithm, further improving the efficiency of neck acupoint recognition.

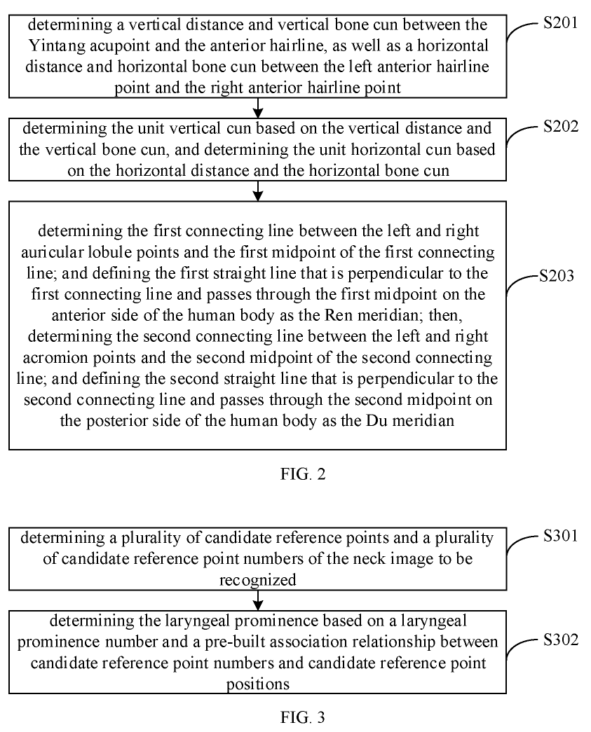

In a specific embodiment of the present disclosure, as shown in FIG. 2, determining a unit vertical cun and a unit horizontal cun of the neck image based on the key points, and determining the Ren meridian and the Du meridian of the neck image based on the feature points in the S101, comprises:.

S201, determining a vertical distance and vertical bone cun between the Yintang acupoint and the anterior hairline, as well as a horizontal distance and horizontal bone cun between the left anterior hairline point and the right anterior hairline point.

The vertical distance and the horizontal distance refer to distances in an image coordinate system where the neck image to be recognized is located, and the horizontal bone cun and the vertical bone cun refer to distances in the bone-degree measurements.

S202, determining the unit vertical cun based on the vertical distance and the vertical bone cun, and determining the unit horizontal cun based on the horizontal distance and the horizontal bone cun.

Specifically, the unit vertical cun is a ratio of the vertical bone cun to the vertical distance, and the unit horizontal cun is a ratio of the horizontal bone cun to the horizontal distance.

S203, determining the first connecting line between the left and right auricular lobule points and the first midpoint of the first connecting line; and defining the first straight line that is perpendicular to the first connecting line and passes through the first midpoint on the anterior side of the human body as the Ren meridian; then, determining the second connecting line between the left and right acromion points and the second midpoint of the second connecting line; and defining the second straight line that is perpendicular to the second connecting line and passes through the second midpoint on the posterior side of the human body as the Du meridian.

By determining the unit horizontal cun and the unit vertical cun, the embodiments of the present disclosure eliminate the adverse effects on acupoint localization resulting from variations in horizontal and vertical distances due to factors such as camera angle, individual anatomical differences, etc, and the neck acupoints are recognized at normalized dimensions, thereby further improving the localization accuracy.

In some embodiments of the present disclosure, as shown in FIG. 3, S102 comprises:

S301, determining a plurality of candidate reference points and a plurality of candidate reference point numbers of the neck image to be recognized.

The candidate reference points include the laryngeal prominence.

It is noted that the laryngeal prominence is crucial as a reference point for the accuracy of subsequent neck acupoint recognition. Therefore, to ensure the accuracy of laryngeal prominence recognition, it is necessary to apply a Poisson reconstruction method or a KinectFusion or DynamicFusion technique to perform three-dimensional reconstruction on three-dimensional point cloud data of the head and neck obtained by the depth camera before S301.

S302, determining the laryngeal prominence based on a laryngeal prominence number and a pre-built association relationship between candidate reference point numbers and candidate reference point positions.

The embodiments of the present disclosure determine, based on the pre-built association relationship between the candidate reference point numbers and the candidate reference point positions, a position of the laryngeal prominence, without the need to locate the laryngeal prominence through parameters such as coordinates. The laryngeal prominence can be located merely based on the candidate reference point numbers, reducing the complexity of laryngeal prominence locating. This can further improve the efficiency of neck acupoint localization.

In a specific embodiment of the present disclosure, the accurate recognition of the laryngeal prominence is crucial for the accurate recognition of the neck acupoints. Therefore, in a specific embodiment of the present disclosure, a plurality of candidate reference points of the neck image to be recognized are determined based on HRNet (High-Resolution Network). The HRNet connects sub-networks of different resolutions in parallel and performs multi-scale fusion among these sub-networks to generate a reliable high-resolution representation. The design of the HRNet enables it to have higher accuracy and efficiency in key point detection tasks.

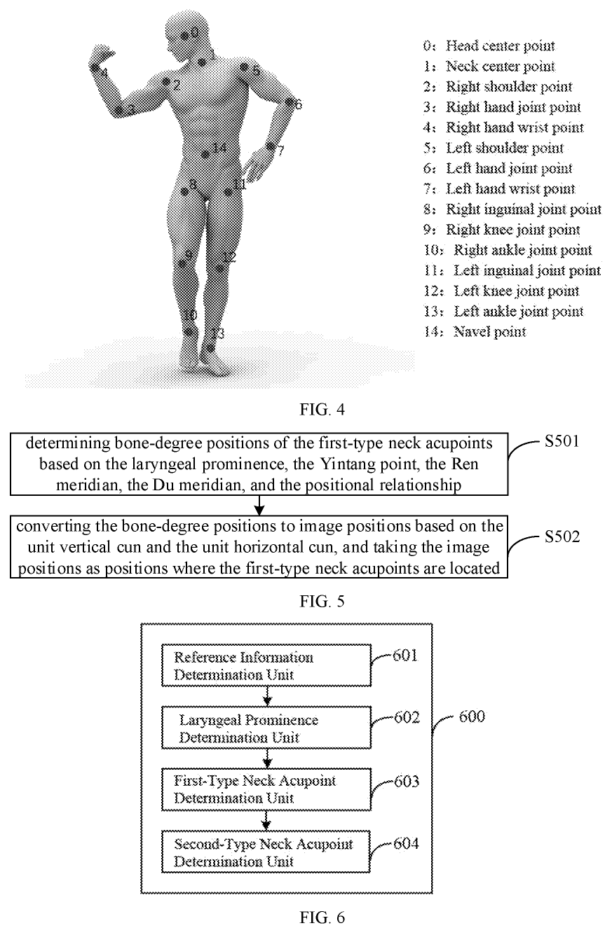

Specifically, when the second detection algorithm is the HRNet, its detection result is as shown in FIG. 4. As can be seen from FIG. 4, the candidate reference point numbered 1 is the laryngeal prominence.

In a specific embodiment of the present disclosure, the positional relationship is a bone-degree positions relationship. In other words, the positional relationship refers to horizontal and vertical cun relationships between each neck acupoint and the laryngeal prominence or between the neck acupoints.

The neck acupoint recognition refers to positions in the actual neck image to be recognized. Therefore, in some embodiments of the present disclosure, as shown in FIG. 5, determining locations of the first-type neck acupoints based on the laryngeal prominence, the Yintang acupoint, the Ren meridian, the Du meridian, the positional relationship, the unit vertical cun and the unit horizontal cun in S103, comprises.

S501, determining bone-degree positions of the first-type neck acupoints based on the laryngeal prominence, the Yintang point, the Ren meridian, the Du meridian, and the positional relationship.

The bone-degree positions refer to positions measured in horizontal cun and vertical cun.

S502, converting the bone-degree positions to image positions based on the unit vertical cun and the unit horizontal cun, and taking the image positions as positions where the first-type neck acupoints are located.

The embodiments of the present disclosure, after determining the bone-degree positions, convert the bone-degree positions to the image positions based on the unit vertical cun and the unit horizontal cun. This can improve the adaptability of the neck acupoints to the neck image to be recognized, facilitating subsequent post-processing operations on the recognized neck acupoints.

It is to be noted that the second-type neck acupoints are also bone-degree positions. The second-type neck acupoints, after being determined, should also be converted to image positions according to the unit vertical cun and the unit horizontal cun.

In some embodiments of the present disclosure, the first-type neck acupoints include Renying acupoint, Futu acupoint, Tiantu acupoint and Tianchuang acupoint that are located on the anterior neck, as well as Yamen and Dazhui that are located on the posterior neck. The positional relationship, comprises:
the Renying acupoint is 1.5 horizontal cun from the laryngeal prominence;
the Futu acupoint is 3 horizontal cun from the laryngeal prominence;
the Tiantu acupoint is located on the Ren meridian and below the laryngeal prominence and is 2 vertical cun from the laryngeal prominence;
the Tianchuang acupoint is 3.5 horizontal cun from the laryngeal prominence;
the Yamen acupoint is located on the Du meridian and is 14.5 vertical cun from the Yintang acupoint;
the Dazhui acupoint is located on the Du meridian and is 18 vertical cun from the Yintang acupoint.

In some embodiments of the present disclosure, the second-type neck acupoints include Lianquan, Qishe, Shuitu, Quepen and Tianding that are located on the anterior neck, as well as Fengfu, Fengchi, Jingbailao and Tianzhu that are located on the posterior neck. The geometric association is as follows:
the Lianquan acupoint is located at the midpoint of the line connecting the chin and the laryngeal prominence; where the chin is the lower box line of the rectangular facial box;
the Qishe acupoint is 1.5 horizontal cun from the Tiantu acupoint;
the Shuitu acupoint is located on the Ren meridian and at the midpoint of the line connecting the Qishe acupoint and the Renying acupoint;
the Quepen acupoint is the intersection point of the extension line of the line connecting the Tiantu acupoint and the Qishe acupoint and a target line segment, where the target line segment is the line segment that is perpendicular to the line connecting the left nipple and the right nipple and passes through the left nipple or the right nipple, where the left nipple and the right nipple can be determined based on open-source models such as Faster R-CNN, SSD, and YOLO;
the Tianding acupoint is located at the midpoint of the line connecting the Futu acupoint and the Quepen acupoint;
the Fengfu acupoint is located above the Yamen acupoint and is 0.5 vertical cun from the Yamen acupoint;
the Fengchi acupoint is 1.3 horizontal cun from the Fengfu acupoint;
the Jingbailao acupoint is located above the Dazhui acupoint and is 2 vertical cun and 1 horizontal cun from the Dazhui acupoint;
the Tianzhu acupoint is located below the Yamen acupoint and is 0.3 vertical cun and 1.3 horizontal cun from the Yamen acupoint.

It is to be noted that the positional relationship between cervical vertebrae is utilized when the position of the Tianzhu acupoint is determined. Specifically, assuming that each cervical vertebra is of the same size, there are 6 cervical vertebrae between the Yamen acupoint and the Dazhui acupoint. It can be known from the above-mentioned positional relationship that the distance between the Yamen acupoint and the Dazhui acupoint is 3.5 vertical cun. Therefore, each cervical vertebra=3.5 vertical cun/6=0.6 vertical cun. The Tianzhu acupoint is half a cervical vertebra away from the Yamen acupoint, so it can be obtained that the distance between the Tianzhu acupoint and the Yamen acupoint is 0.3 vertical cun.

During the process of determining the positional relationship, the embodiments of the present disclosure reference the positions of cervical vertebrae of a human body, further ensuring the accuracy of the positional relationship.

The bone-degree measurement used in the foregoing embodiments is the bone-degree between the Yintang acupoint and the anterior hairline serving as a reference for neck acupoint recognition, and the bone-degree may also be the bone-degree between the anterior hairline and the posterior hairline. Therefore, to further verify the accuracy of the recognized neck acupoints, in some embodiments of the present disclosure, the neck acupoint recognition method further includes:
determining the posterior hairline based on the anterior hairline, wherein the posterior hairline is 12 vertical cun from the anterior hairline;
verifying whether the positional relationships are accurate based on the posterior hairline and the Yamen point, Dazhui point, and Fengfu point.

Specifically, the positional relationship is verified by the Yamen acupoint, the Dazhui acupoint and the Fengfu acupoint, that are determined based on the posterior hairline, and it is judged whether these acupoints are coincident with the Yamen acupoint, the Dazhui acupoint and the Fengfu acupoint that are determined based on the Yintang acupoint. If they are coincident, the positional relationship is determined to be accurate; otherwise, it is not accurate.

Specifically, the Yamen point, Dazhui point, and Fengfu point are determined based on the posterior hairline and the positional relationships, and it is judged whether they coincide with the Yamen point, Dazhui point, and Fengfu point determined based on the Yintang point. If they coincide, the positional relationships are determined to be accurate; otherwise, they are not accurate.

In a specific embodiment of the present disclosure, the Yamen acupoint is located above the posterior hairline and is 0.5 vertical cun from the posterior hairline, the Dazhui acupoint is located below the posterior hairline and is 3 vertical cun from the posterior hairline, and the Fengfu acupoint is located below the posterior hairline and is 1 vertical cun from the posterior hairline.

In summary, the embodiments of the present disclosure further verify the accuracy of the neck acupoints through the posterior hairline.

To better implement the neck acupoint recognition method in the embodiments of the present disclosure, based on the neck acupoint recognition method, accordingly, an embodiment of the present disclosure further provides a neck acupoint recognition device. As shown in FIG. 6, the neck acupoint recognition device 600 comprises:
a reference information determination unit 601, configured to determine the key points associated with horizontal and vertical cun and feature points associated with meridians, of a neck image to be recognized, determine the unit vertical cun and the unit horizontal cun of the neck image based on the key points, and determine the Ren meridian and the Du meridian of the neck image based on the feature points, wherein the key points include the Yintang acupoint;
a laryngeal prominence determination unit 602, configured to determine a laryngeal prominence of the neck image to be recognized;
a first-type neck acupoint determination unit 603, configured to determine a positional relationship between the first-type neck acupoints and a set of anatomical landmarks, where the set of anatomical landmarks includes the laryngeal prominence, the Yintang acupoint, the Ren meridian, and the Du meridian, and determine locations of the first-type neck acupoints based on the laryngeal prominence, the Yintang acupoint, the Ren meridian, the Du meridian, the positional relationship, the unit vertical cun and the unit horizontal cun;
a second-type neck acupoint determination unit 604, configured to determine a geometric association between the first-type neck acupoints and second-type neck acupoints, and determine the second-type neck acupoints based on the geometric association and the first-type neck acupoints.

The neck acupoint recognition device 600 provided in the above-mentioned embodiment can implement the technical solutions described in the above-mentioned neck acupoint recognition method embodiments. The specific implementation principles of the above-mentioned modules or units can be seen in the corresponding content in the above-mentioned neck acupoint recognition method embodiments, which will not be elaborated here.

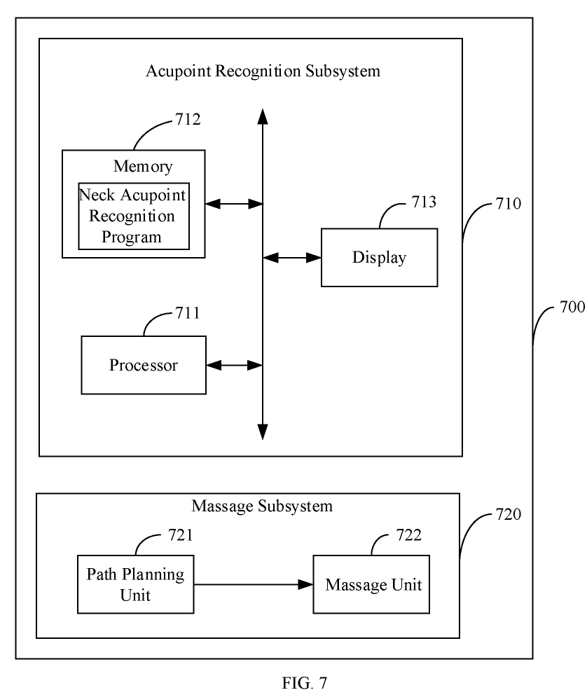

As shown in FIG. 7, the present disclosure also correspondingly provides a massage robot 700. The massage robot 700 includes an acupoint recognition subsystem 710 configured to determine neck acupoints and a massage subsystem 720 configured for performing massage based on the neck acupoints. The acupoint recognition subsystem 710 includes a processor 711, a memory 712, and a display 713. FIG. 7 merely shows some components of the massage robot 700. However, it should be understood that not all shown components are required to be implemented, and more or fewer components can be implemented alternatively.

In some embodiments, the memory 712 may be an internal storage unit of the acupoint recognition subsystem 710, for example, a hard disk or an internal memory of the acupoint recognition subsystem 710. In other embodiments, the memory 712 may also be an external storage device of the acupoint recognition subsystem 710, for example, a plug-in hard disk, a smart media card (SMC), a secure digital (SD) card, a flash card, or the like that are equipped on the acupoint recognition subsystem 710.

Further, the memory 712 may also include both an internal storage unit and an external storage device of the acupoint recognition subsystem 710. The memory 712 is configured to store application software and various data for installing the acupoint recognition subsystem 710.

In some embodiments, the processor 711 may be a central processing unit (CPU), a microprocessor, or other data processing chips and is configured to run a program code stored in the memory 712 or process data, for example, the neck acupoint recognition method in the present disclosure.

In some embodiments, the display 713 may be an LED display, a liquid crystal display, a touch liquid crystal display, an organic light-emitting diode (OLED) touch device, or the like. The display 713 is configured to display information of the acupoint recognition subsystem 710 and to display a visualized user interface. The components 711-713 of the acupoint recognition subsystem 710 communicate with each other through a system bus.

In some embodiments of the present disclosure, the processor 711, when executing a neck acupoint recognition program in the memory 712, can implement the following steps:
determining, for a neck image to be recognized, key points associated with horizontal and vertical cun and feature points associated with meridians, determining a unit vertical cun and a unit horizontal cun of the neck image to be recognized based on the key points, and determining a Ren meridian and a Du meridian of the neck image to be recognized based on the feature points, where the key points include a Yintang acupoint;
determining a laryngeal prominence of the neck image to be recognized;
determining a positional relationship between first-type neck acupoints and a set of anatomical landmarks, where the set of anatomical landmarks includes the laryngeal prominence, the Yintang acupoint, the Ren meridian, and the Du meridian, and determining locations of the first-type neck acupoints based on the laryngeal prominence, the Yintang acupoint, the Ren meridian, the Du meridian, the positional relationship, the unit vertical cun and the unit horizontal cun;
determining a correlation between the first-type neck acupoints and second-type neck acupoints, and determining the second-type neck acupoints based on both the correlation and the first-type neck acupoints.

It should be understood that the processor 711, when executing the neck acupoint recognition program in the memory 712, can also implement other functions in addition to the above functions. The specific details can be seen in the previous descriptions of the related method embodiments.

As shown in FIG. 7, the massage subsystem 720 includes a path planning unit 721 and a massage unit 722. The path planning unit 721 is configured to plan a path according to positions of moxibustion needles in the massage unit 722 and coordinates of the acupoints and generate a planned path. The massage unit 722 moves the moxibustion needles to positions where the acupoints are located according to the planned path.

The embodiments of the present disclosure, by arranging the massage robot 700, can replace a doctor in performing long-term massage therapy, thereby reducing the burden on the doctor and lowering medical consumption.

In another aspect, an embodiment of the present disclosure further provides a digital human, including neck acupoints that are used for a virtual neck model and are marked in the virtual neck model, where the neck acupoints are determined based on a neck acupoint recognition method, where the neck acupoint recognition method is the neck acupoint recognition method in any one of the above-mentioned embodiments.

The embodiment of the present disclosure, by arranging the digital human, can achieve training purposes through the digital human, for example, training in cognition of neck acupoints, or the like.

It can be understood by those skilled in the art that all or part of the processes for implementing the above-mentioned embodiment methods can be completed by instructing relevant hardware (such as a processor and a controller) through a computer program. The computer program can be stored on a computer-readable storage medium. The computer-readable storage medium is a magnetic disk, an optical disk, a read-only memory, or a random access memory, or the like.

The neck acupoint recognition method and device, the massage robot, and the digital human provided by the present disclosure have been described in detail above. Specific examples have been used herein to illustrate the principles and embodiments of the present disclosure. The descriptions of the above embodiments are merely used for helping to understand the method and core ideas of the present disclosure. Meanwhile, for those skilled in the art, based on the ideas of the present disclosure, changes can be made to the specific embodiments and application ranges. In conclusion, the content of this specification should not be understood as limiting the present disclosure.

What is claimed is:

1. A neck acupoint recognition method, comprising:
determining, for a neck image to be recognized, key points associated with horizontal and vertical cun and feature points associated with meridians, determining a unit vertical cun and a unit horizontal cun of the neck image to be recognized based on the key points, and determining the Ren meridian and the Du meridian of the neck image to be recognized based on the feature points, wherein the key points comprise the Yintang acupoint;
determining a laryngeal prominence of the neck image to be recognized;
determining a positional relationship between first-type neck acupoints and a set of anatomical landmarks, wherein the set of anatomical landmarks comprises the laryngeal prominence, the Yintang acupoint, the Ren meridian, and the Du meridian, and determining locations of the first-type neck acupoints based on the laryngeal prominence, the Yintang acupoint, the Ren meridian, the Du meridian, the positional relationship, the unit vertical cun and the unit horizontal cun;
determining a geometric association between the first-type neck acupoints and second-type neck acupoints, and determining the second-type neck acupoints based on the geometric association and the first-type neck acupoints;
wherein the first-type neck acupoints comprise Renying, Futu, Tiantu and Tianchuang that are located on an anterior neck, as well as Yamen and Dazhui that are located on a posterior neck; wherein the positional relationship comprises:
the Renying acupoint is 1.5 horizontal cun from the laryngeal prominence;
the Futu acupoint is 3 horizontal cun from the laryngeal prominence;
the Tiantu acupoint is located on the Ren meridian and below the laryngeal prominence and is 2 vertical cun from the laryngeal prominence;
the Tianchuang acupoint is 3.5 horizontal cun from the laryngeal prominence;
the Yamen acupoint is located on the Du meridian and is 14.5 vertical cun from the Yintang acupoint;
the Dazhui acupoint is located on the Du meridian and is 18 vertical cun from the Yintang acupoint;
wherein the second-type neck acupoints comprise Lianquan, Qishe, Shuitu, Quepen and Tianding that are located on the anterior neck, as well as Fengfu, Fengchi, Jingbailao and Tianzhu that are located on the posterior neck; wherein the correlation relationship is as follows:
the Lianquan acupoint is located at the midpoint of the line connecting the chin and the laryngeal prominence; wherein the chin is the lower box line of the rectangular facial box in the neck image to be recognized;
the Qishe acupoint is 1.5 horizontal cun from the Tiantu acupoint;
the Shuitu acupoint is located on the Ren meridian and at the midpoint of the line connecting the Qishe acupoint and the Renying acupoint;
the Quepen acupoint is the intersection point of the extension line of the line connecting Tiantu acupoint and Qishe acupoint and a target line segment, wherein the target line segment is the line segment that is perpendicular to the line connecting the left nipple and the right nipple and passes through the left nipple or the right nipple;
the Tianding acupoint is located at the midpoint of the line connecting the Futu acupoint and the Quepen acupoint;
the Fengfu acupoint is located above the Yamen acupoint and is 0.5 vertical cun from the Yamen acupoint;
the Fengchi acupoint is 1.3 horizontal cun from the Fengfu acupoint;
the Jingbailao acupoint is located above the Dazhui acupoint and is 2 vertical cun and 1 horizontal cun from the Dazhui acupoint;
the Tianzhu acupoint is located below the Yamen acupoint and is 0.3 vertical cun and 1.3 horizontal cun from the Yamen acupoint.

2. The neck acupoint recognition method according to claim 1, the key points further comprise a left anterior hairline point and a right anterior hairline point that are associated with the horizontal cun, as well as an anterior hairline associated with the vertical cun; wherein the feature points comprise a left auricular lobule point, a right auricular lobule point, a left acromion point and a right acromion point; then, determining the key points associated with the horizontal and vertical cun and the feature points associated with the meridians of the neck image to be recognized, comprises:
detecting the neck image to be recognized to obtain a plurality of candidate key points, and determining, based on a mapping relationship between candidate key point numbers and the candidate key points, the left anterior hairline point, the right anterior hairline point, a first target point and a second target point that are associated with the Yintang acupoint, the left auricular lobule point, the right auricular lobule point, the left acromion point and the right acromion point from the plurality of candidate key points;
determining the midpoint of the first target point and the second target point as the Yintang acupoint;
determining the rectangular facial box in the neck image to be recognized, and defining the upper boundary of the rectangular facial box as the anterior hairline.

3. The neck acupoint recognition method according to claim 2, determining a unit vertical cun and a unit horizontal cun of the neck image based on the key points, and determining a Ren meridian and a Du meridian of the neck image based on the feature points, comprises, comprises:
determining a vertical distance and vertical bone cun between the Yintang acupoint and the anterior hairline, as well as a horizontal distance and horizontal bone cun between the left anterior hairline point and the right anterior hairline point;
determining the unit vertical cun based on the vertical distance and the vertical bone cun, and determining the unit horizontal cun based on the horizontal distance and the horizontal bone cun;
determining a first connecting line between the left and right auricular lobule points and a first midpoint of the first connecting line; and defining a first straight line that is perpendicular to the first connecting line and passes through the first midpoint on an anterior side of a human body as the Ren meridian; then, determining a second connecting line between the left and right acromion points and a second midpoint of the second connecting line; and defining a second straight line that is perpendicular to the second connecting line and passes through the second midpoint on a posterior side of the human body as the Du meridian.

4. The neck acupoint recognition method according to claim 1, determining the laryngeal prominence of the neck image to be recognized, comprises:
determining a plurality of candidate reference points and a plurality of candidate reference point numbers of the neck image to be recognized;
determining the laryngeal prominence based on a laryngeal prominence number and a pre-built association relationship between candidate reference point numbers and candidate reference point positions.

5. The neck acupoint recognition method according to claim 1, wherein the positional relationship is a bone-degree positions relationship; then, determining locations of the first-type neck acupoints based on the laryngeal prominence, the Yintang acupoint, the Ren meridian, the Du meridian, the positional relationship, the unit vertical cun and the unit horizontal cun, comprises:
determining bone-degree positions of the first-type neck acupoints based on the laryngeal prominence, the Yintang point, the Ren meridian, the Du meridian, and the positional relationship;
converting the bone-degree positions to image positions based on the unit vertical cun and the unit horizontal cun, and taking the image positions as positions where the first-type neck acupoints are located.

6. A neck acupoint recognition device, comprising:
a reference information determination unit, configured to determine the key points associated with horizontal and vertical cun and feature points associated with meridians, of a neck image to be recognized, determine the unit vertical cun and the unit horizontal cun of the neck image based on the key points, and determine the Ren meridian and the Du meridian of the neck image based on the feature points, wherein the key points include the Yintang acupoint;
a laryngeal prominence determination unit, configured to determine a laryngeal prominence of the neck image to be recognized;
a first-type neck acupoint determination unit, configured to determine a positional relationship between the first-type neck acupoints and a set of anatomical landmarks, wherein the set of anatomical landmarks comprises the laryngeal prominence, the Yintang acupoint, the Ren meridian, and the Du meridian, and determine locations of the first-type neck acupoints based on the laryngeal prominence, the Yintang acupoint, the Ren meridian, the Du meridian, the positional relationship, the unit vertical cun and the unit horizontal cun;
a second-type neck acupoint determination unit, configured to determine a geometric association between the first-type neck acupoints and second-type neck acupoints, and determine the second-type neck acupoints based on the geometric association and the first-type neck acupoints;
wherein the first-type neck acupoints comprise Renying, Futu, Tiantu and Tianchuang that are located on an anterior neck, as well as Yamen and Dazhui that are located on a posterior neck; wherein the positional relationship comprises:
the Renying acupoint is 1.5 horizontal cun from the laryngeal prominence;
the Futu acupoint is 3 horizontal cun from the laryngeal prominence;
the Tiantu acupoint is located on the Ren meridian and below the laryngeal prominence and is 2 vertical cun from the laryngeal prominence;
the Tianchuang acupoint is 3.5 horizontal cun from the laryngeal prominence;
the Yamen acupoint is located on the Du meridian and is 14.5 vertical cun from the Yintang acupoint;
the Dazhui acupoint is located on the Du meridian and is 18 vertical cun from the Yintang acupoint;
wherein the second-type neck acupoints comprise Lianquan, Qishe, Shuitu, Quepen and Tianding that are located on the anterior neck, as well as Fengfu, Fengchi, Jingbailao and Tianzhu that are located on the posterior neck; wherein the geometric association is as follows:
the Lianquan acupoint is located at the midpoint of the line connecting the chin and the laryngeal prominence; wherein the chin is the lower box line of the rectangular facial box in the neck image to be recognized;
the Qishe acupoint is 1.5 horizontal cun from the Tiantu acupoint;
the Shuitu acupoint is located on the Ren meridian and at the midpoint of the line connecting the Qishe acupoint and the Renying acupoint;
the Quepen acupoint is the intersection point of the extension line of the line connecting the Tiantu acupoint and the Qishe acupoint and a target line segment, wherein the target line segment is the line segment that is perpendicular to the line connecting the left nipple and the right nipple and passes through the left nipple or the right nipple;
the Tianding acupoint is located at the midpoint of the line connecting the Futu acupoint and the Quepen acupoint;
the Fengfu acupoint is located above the Yamen acupoint and is 0.5 vertical cun from the Yamen acupoint;
the Fengchi acupoint is 1.3 horizontal cun from the Fengfu acupoint;
the Jingbailao acupoint is located above the Dazhui acupoint and is 2 vertical cun and 1 horizontal cun from the Dazhui acupoint; and
the Tianzhu acupoint is located below the Yamen acupoint and is 0.3 vertical cun and 1.3 horizontal cun from the Yamen acupoint.

7. A massage robot, comprising an acupoint recognition subsystem configured to determine neck acupoints and a massage subsystem configured to perform massage based on the neck acupoints, wherein the acupoint recognition subsystem comprises a memory and a processor, wherein
the memory is configured to store a program;
the processor is coupled with the memory and configured to execute the program stored in the memory to implement the steps of the neck acupoint recognition method according to claim 1.

8. A digital human, comprising neck acupoints that are used for a virtual neck model and are marked in the virtual neck model, wherein the neck acupoints are determined based on a neck acupoint recognition method, wherein the neck acupoint recognition method is the neck acupoint recognition method according to claim 1.
